(19) United States
(12) Patent Application Publication
(10) Pub. No.: US 2009/0317466 A1
(43) Pub. Date: Dec. 24, 2009

(54) Fixed dose pharmaceutical composition comprising hyroxyurea and didanosine

(76) Inventor: Franco Lori, Bethesda, MD (US)

Correspondence Address: THE LAW OFFICES OF VALERIE E. LOOPER, 11726 LIGHTFALL COURT, COLUMBIA, MD, 21044, US

(21) Appl. No.: 12/214,372

(22) Filed: Jun. 18, 2008

(51) Int. Cl. A61K 9/28; A61K 31/708; A61P 31/18

(52) U.S. Cl. 424/474; 514/45

(57) ABSTRACT

Pharmaceutical composition, containing fixed doses of hydroxyurea and didanosine, a method of manufacturing such composition, and to the use of the composition for the treatment of retroviral infections.

### FIELD OF THE INVENTION

The present invention relates to a pharmaceutical composition containing hydroxyurea (aka hydroxycarbamide or HU) and 2′,3′-dideoxyinosine (aka didanosine or ddI), to a method for manufacturing of such composition, and to the use of the composition for the treatment of infections caused by reverse transcriptase dependent viruses.

### BACKGROUND OF THE INVENTION

Human immunodeficiency virus (HIV) is a reverse transcriptase dependent virus that was first identified as the causative agent of AIDS in 1983. The large pool of HIV carriers makes the development of effective antiviral treatments a medical priority. For twenty-five years, many compounds have been suggested as pharmaceuticals useful in the treatment of HIV, both as single agents and, more and more, in combination.

Combination therapy is essential for the treatment of HIV/AIDS. The goals of HIV therapy are to maximally and durably suppress the virus, to allow recovery of the immune system and reduce the emergence of HIV resistance. At least three active drugs, usually from two different classes, are required to suppress the virus, allow recovery of the immune system, and reduce the emergence of HIV resistance. In the United States and developing countries, simplified HIV regimens in the form of Fixed Dose Combinations (FDCs) may facilitate distribution and improve patient compliance.

The goal of having FDC products is to simplify regimens to allow for easier distribution and improved patient compliance, particularly in resource poor settings. Proposed combination products should be relatively well tolerated and easy to administer while providing potency and a sufficient barrier to the emergence of drug resistance. When developing FDCs, the US FDA, (Guidance for Industry: Fixed Dose Combination and Co-Packaged Drug Products for Treatment of HIV) recommends that the products have the following important characteristics:
Contain two or more components of a fully suppressive regimen
Require a once or twice daily administration
Be recommended as a preferred or alternate regimen (or regimen component) in treatment guidelines
Have clinical efficacy and safety data that support use of the combination
Be commonly used in treatment-naive patients
Have drug interaction and toxicity profiles that allow for concomitant dosing

When considering proposed FDCs, investigators should take into account the required dosing frequency of each of the components. Each of the components of an FDC should preferably have an identical dosing frequency and similar food instructions. Therefore, investigators should consider differences in food instructions between individual components when developing FDC products.

In a report of a meeting held at the WHO headquarters in Geneva in 2003 (FIXED-DOSE COMBINATIONS FOR HIV/AIDS, TUBERCULOSIS, AND MALARIA, Report of a meeting held 16-18 Dec. 2003, Geneva, World Health Organization) additional benefits of FDC combination therapy were defined as being:
Increase patient compliance to treatment
Delay the development of resistance
Lower the total cost, including production, storage, transport, dispensing and other health system costs
Reduce the risk of medication errors by prescribers, dispensers or patients themselves
Simplify and increase security of supply systems
Facilitate patient counselling and education, reduce waiting time

Current therapeutic approaches to reduce the replicative capacity of HIV-1 attempt to drive down viral replication by acting upon multiple steps in the natural life-cycle of the virus. A more complete understanding of the influence of chronic immune activation in the presence of HIV disease has opened another route for controlling viral replication.

Hydroxyurea is a compound which was originally used as an anticancer agent. It inhibits ribonucleotide reductase, and is used as an antiviral agent (J. Med. Chem. Vol. 28, 1985, pages 1103-06 Tang et al ‘Optimization of the Schiff Bases of N-hydroxy-N′ aminoguanidines as Anticancer and Antiviral Agents’). Hydroxyurea is a member of the relatively new family of anti-HIV drugs called ‘virostatics’, having both antiviral (directly suppressing HIV) and cytostatic (preventing immune system over-activation) activity. Data from in vitro and clinical studies have proven that hydroxyurea-based regimens are effective options for patients suffering from an HIV infection.

Concerns over hydroxyurea toxicity have however limited its use. The major side effect of hydroxyurea is suppression of the bone marrow. Bone marrow suppression can be revealed on laboratory tests as low counts for red blood cells (anemia), white blood cells (neutropenia, leukopenia), platelets (thrombocytopenia), or as low numbers of all types of blood cells (pancytopenia). For people already having bone marrow problems, hydroxyurea is not recommended. Less frequent side effects include hair loss, anorexia, nausea, vomiting, diarrhea and constipation. Rashes, particularly on the face, have also been reported. Hydroxyurea has been shown to cause birth defects in animals and should not be taken by pregnant women. Because hydroxyurea is classified as a mutagen, accidental exposure to the drug, particularly the powder, should be avoided.

Hydroxyurea is marketed under two brand names in the US and Europe by Bristol-Myers Squibb (BMS) as capsules filled with powder. The qualitative composition of the US product Droxia® capsules, a product carrying US orphan drug status for the treatment of sickle cell anemia, (hydroxyurea 200, 300 and 400 mg/capsule) is the following: hydroxyurea, citric acid, gelatin, lactose, magnesium stearate, sodium phosphate, titanium dioxide and colorants of capsules)

In Europe, the capsules are only available in one strength, 500 mg, and the qualitative composition of the product Hydrea® capsules is the following: hydroxyurea, citric acid, disodium phosphate, lactose monohydrate, magnesium stearate, gelatin, titanium dioxide and colorants.

2′,3′-dideoxyinosine (didanosine) belongs to the chemical family of nucleoside phosphate analogues. Like other nucleoside analogues, didanosine is an inhibitor of reverse transcriptase, the enzyme used by the human immunodeficiency virus to convert viral RNA into proviral DNA. As a nucleoside inhibitor, didanosine both competes with the endogenous nucleotide, deoxyadenosine triphosphate (dATP) and, upon incorporation into the growing proviral DNA strand, causes chain termination. Didanosine is currently marketed as Videx® EC which is a capsule containing enteric coated beadlets. It is available in various strengths (125, 200, 250 & 400 mg). The beadlets consist of carboxymethylcellulose sodium, diethyl phthalate, methacrylic acid copolymer, sodium hydroxide, sodium starch glycolate, talc and colloidal silicon dioxide. The 400 mg capsules also contain gelatin, sodium lauryl sulphate and titanium dioxide.

The combined use of separate hydroxyurea and didanosine tablets or capsules is currently known. Studies have reported that taking hydroxyurea together with didanosine produces a stronger anti-HIV effect than taking didanosine alone. Initial enthusiasm concerning the use of hydroxyurea and didanosine in the treatment of HIV disease centered upon the action of hydroxyurea to potentiate didanosine's antiviral activity and the ability of the combination to restore didanosine sensitivity in resistant viral populations. However, toxicities also emerged that resulted in a significant decline in the combination's use.

Based upon observations in clinical trials conducted in the late 1990s, the combination of hydroxyurea and didanosine was generally perceived to be too toxic for clinical use. Indeed, when used in combination with stavudine (Zerit®, d4T) and with the relatively high doses of hydroxyurea and didanosine used at that time, the combination appeared to increase both the incidence and severity of nucleoside toxicity. In fact, the reason most often cited for discontinuing the combination, both in clinical trials and in practice, was peripheral neuropathy (nerve toxicity). More serious toxicities, including several cases of fatal pancreatitis, were also seen when high doses of hydroxyurea were administered with full-dose didanosine and stavudine.

There were two cases of fatal pancreatitis in the AIDS Clinical Trials Group (ACTG) 5025 trial. In this study, subjects who were maximally suppressed on an AZT-3TC-based HAART regimen were randomized to continue the regimen or to switch to stavudine-didanosine-indinavir (a protease inhibitor) with or without high-dose hydroxyurea (1,200 mg). Although two cases of fatal pancreatitis occurred on the high-dose hydroxyurea arm, the overall incidence of treatment-related pancreatic toxicity was the same for both stavudine-didanosine-indinavir arms, with or without high-dose hydroxyurea. In a subsequent meta-analysis of NIH-coordinated ACTG trials, the regimen with the highest risk of pancreatitis was stavudine-didanosine-indinavir and the risk was independent of the addition of hydroxyurea. The authors concluded that hydroxyurea does not increase the risk of pancreatitis compared to didanosine alone when combined with stavudine.

Hematological toxicities were also reported in trials investigating high doses of hydroxyurea (i.e., >1,000 mg/day). In the ACTG 307 trial, cases of anemia, thrombocytopenia and neutropenia were reported, but these occurred almost exclusively in the arm receiving the highest dose of hydroxyurea (i.e., 1,500 mg/day).

Also, when using separate tablets and capsules, as in the combined hydroxyurea and didanosine therapy known in the prior art, patient compliance (crucial for a successful treatment) cannot always be attained. Therefore, patient compliance could be improved significantly by using a single fixed combination product instead of taking separate hydroxyurea capsules and didanosine tablets several times a day. It is, however, an accepted fact that Fixed Dose Combination (FDC) products are treated as a new drug by Health Authorities because, by combining two or more drugs, the safety, efficacy, and bioavailability of the individual Active Pharmaceutical Ingredients (API) may change. Therefore, a need exists for a new pharmaceutical formulation that yields enhanced control of both the relative and absolute amounts of each active ingredient.

Furthermore, it is very difficult to adjust the absorption of multiple active agents from one and the same pharmaceutical fixed dose composition. Usually in practice, the absorption of one of the active agents may decrease while that of the other increases. When selecting the pharmaceutical excipients, disintegrants and/or other auxiliary agents to be used in pharmaceutical compositions in combination with several active agents, numerous factors have to be considered (e.g. the chemical and physical characteristics of the active agents and the auxiliary agents, the bioavailabilities of the active agents, the method of preparing the composition, the stability of the composition, etc.). Even when these factors are considered, at this stage of the art, any given composition must be tested before conclusions can be drawn about the formulation that best supports product efficacy.

The inventors gained a better understanding of the factors that led to the high rates of toxicity reported in early clinical trials of the hydroxyurea-didanosine combination. These factors include:
The combination with stavudine (Zerit®-d4T) led to increased toxicity.

Stavudine toxicities have been shown to be dose sensitive with increased exposure to the drug's active moiety, d4T-triphosphate, resulting in increased toxicities.

Due to overlapping toxicities, the combination of stavudine and didanosine is no longer a recommended strategy in the treatment of HIV disease. Additionally, intracellular concentrations of this active moiety, d4T-triphosphate, are elevated in the presence of hydroxyurea due to increased activity of thymidine kinase;
Lack of Weight-based Dosing Adjustments for stavudine and didanosine. Doses of both stavudine and didanosine are recommended to be reduced in patients weighing less than 60 kg. As this dose reduction is unique to these two drugs, the reductions were often not made, resulting to increased exposure to both agents;
Higher peak blood concentrations (Cmax) for didanosine as once-daily (QD) chewable tablets replaced twice-daily (BID) chewable tablets. Higher didanosine Cmax were experienced as QD chewable tablets replaced BID dosing. It is an accepted observation that increased toxicity of pharmaceutical agents often corresponds to increased levels of drug exposure. In an attempt to increase patient convenience, the dosing of didanosine underwent a change from 200 mg BID to 400 mg QD (in patients weighing >60 kg), including its use in regimens containing hydroxyurea. Prior to the introduction of enterically coated didanosine, this was accomplished by administering the full daily dose at one time using Videx® chewable tablets. As these tablets were immediate release, the resulting Cmax when taking 400 mg QD was in excess of that seen when the agent was administered in two divided doses of 200 mg each.
Increasing Doses of Hydroxyurea. During the late 1990s, the dose of hydroxyurea was being raised in an attempt to further enhance the antiviral activity of the HU-ddI combination. Initially dosed at 500 mg BID, doses were increased to 1,000 mg QD (resulting in a higher Cmax) and, upon availability of Droxia, 1,200 mg, 1,500 mg and higher QD. Paradoxically, the efficacy of the combination was reduced at higher doses of hydroxyurea due to an increase in toxicity (decreased tolerability) and the fact that the agent becomes cytotoxic (i.e., is no longer cytostatic) at higher doses, resulting in a loss of CD4 cells in the majority of patients.
Didanosine Potentiation of Hydroxyurea. Findings both in vitro and in vivo now suggest that, through an as yet unidentified mechanism of action, didanosine actually potentiates the ability of hydroxyurea to decrease the intracellular concentration of dATP, the natural deoxynucleotide (dNTP) with which didanosine's active metabolite (ddATP) competes. Although not widely recognized at the time the hydroxyurea-didanosine combination was determined to be toxic, this potentiation would have been occurring against a background of the higher doses of hydroxyurea that were being investigated, potentially adding to the toxicity of the combination.
Increased bioavailability of hydroxyurea in the fasting state. The inventors have discovered that the absorption of hydroxyurea is significantly reduced and delayed in the presence of food. As a result, when equivalent doses of hydroxyurea are given in the fasting and fed states, higher exposure to hydroxyurea results in the fasting state. As didanosine can only be given on an empty stomach, hydroxyurea was also given at the same time (i.e., also in the fasting state) in the interest of increasing patient compliance. Thus, increased hydroxyurea exposures were experienced—again, against a background of increasing doses.

The inventors recognized that if these factors were avoided they could reduce toxicity and increase the tolerability of the combination, and so ensure the advantage of the combined used of didanosine and hydroxyurea for larger numbers of patients.

### SUMMARY OF THE INVENTION

The inventors have found an improved pharmaceutical composition, containing hydroxyurea and didanosine, a method of manufacturing such composition, and to the use of the composition for the treatment of retroviral infections, such as HIV.

These results form the basis of a new phase in the clinical development of the hydroxyurea-didanosine combination in which advantage is taken of the synergistic interaction between hydroxyurea and didanosine and their mechanisms of action, combined with hydroxyurea's immunomodulating properties, the activity of hydroxyurea to induce a natural blockade of cell cycle progression, and the combination's favorable resistance profile. The inventors have now found unexpectedly that hydroxyurea can be effective at lower doses when taken by a patient in a fasted state. This regimen allows lower doses of each drug than were previously prescribed, minimizing the complexity, toxicity and cost of treatment while maintaining, or even improving, therapeutic efficacy. For some patients, this may enable the further lowering of the dose of didanosine as well.

### DETAILED DESCRIPTION OF THE INVENTION

The hydroxyurea doses according to the present invention are all less than the 1,000 mg or more per day often administered in earlier trials of the combination. The compositions according to the present invention preferably comprise 200 to 900 mg hydroxyurea and 200 to 400 mg didanosine. In a preferred embodiment, the composition comprises 200 mg didanosine QD, plus 300 mg hydroxyurea QD, 200 mg didanosine QD plus hydroxyurea 600 mg QD, 200 mg didanosine QD plus hydroxyurea 900 mg QD, 400 mg didanosine QD plus hydroxyurea 300 mg QD, 400 mg didanosine QD plus hydroxyurea 600 mg QD. Preferred compositions comprise didanosine 200 mg QD plus hydroxyurea 900 mg QD, or 200 mg didanosine QD and hydroxyurea 825 mg QD, or 325 mg didanosine QD and hydroxyurea 637.5 mg QD.

From a patient-management perspective, it is preferable to dose anti-HIV medications QD, or once-daily (RIGHT 702: AIDS Res Hum Retroviruses. April 2005;21(4):263-72).

It was demonstrated that QD dosing of hydroxyurea in combination with didanosine provides efficacy equivalent to administering the same daily doses given twice daily. 600 mg daily dose of hydroxyurea (combined with a 400 mg daily dose of didanosine) demonstrated better antiviral activity than did higher hydroxyurea doses, together with a greater CD4+T-cell count increase and fewer adverse events. Therefore, 600 mg hydroxyurea QD is preferably selected as the highest hydroxyurea dose, when administered in combination with 400 mg didanosine QD.

Although the pharmaceutical composition according to the present invention can have any form (such as tablets, capsules, powders, etc.) to be used simultaneously, in order to obtain a higher patient compliance, the composition is preferably a fixed dose composition. The applicants have found a particularly interesting way to combine increased efficacy of hydroxyurea, better patient compliance and minimization of exposure to hydroxyurea by combining the active ingredients in single fixed dose composition.

Preferably, the fixed dose composition has the form of a capsule (or any other suitable form providing an outer casing) comprising one or more separately formulated pharmaceutical dosages of hydroxyurea and didanosine.

The capsule can be any suitable capsule made from a suitable material (gelatin, HPMC, starch). Preferably, the capsule is for example a size 0, elongated, opaque white hard gelatin capsule.

In a preferred embodiment, the one or more separately formulated pharmaceutical dosages consist of one or more tablets containing hydroxyurea and didanosine. Commercially available pharmaceutical hydroxyurea (Hydrea®/Droxia®) is marketed as a powder contained in capsules. Therefore, the patient instructions contain warnings in order to avoid contact with the hydroxyurea powder. The formulation according to the present invention is advantageous as the use of hydroxyurea in tablet form with low friability avoids the dustiness of the powder, and, therefore, has an improved safety profile. Additionally, formulating hydroxyurea into a tablet makes it possible to apply a coating thereby reducing the exposure risk even more.

According to the present invention, the didanosine tablets in the fixed dose combination product can be coated or not. Historically, an increase in didanosine compliance was associated with the implementation of QD dosing using the immediate release, chewable tablets. Originally approved as a twice-a-day drug based upon didanosine's plasma half-life, the drug was administered as 200 mg given twice daily (125 mg BID for patients weighing less than 60 kg). As the intracellular half-life of dideoxyadenosine triphosphate (the active didanosine moiety) would support once-daily dosing, practitioners began utilizing didanosine at 400 mg once-daily (250 mg QD for subjects weighing less than 60 kg) using the immediate release chewable tablets. As it has been suggested that greater Cmax, that is, peak plasma concentrations, of didanosine may have been related to increased side effects, the originator company developed an enterically-coated didanosine product to alter the drug's release, or pharmacokinetic profile, and decrease toxicities associated with the high level of buffer contained in four 100 mg didanosine tablets.

The commercially available hydroxyurea tablets contain citric acid. In a preferred embodiment the pharmaceutical composition according to the present invention does not contain the stabilizer citric acid, as this may interact with the enteric coating of the didanosine tablet. Surprisingly, the inventors found that the stability of hydroxyurea was not significantly affected by the removal of citric acid.

The excipients used in accordance with the present invention may be all commercially available excipients.

In an embodiment of the present invention, the didanosine part of the composition comprises didanosine and one or more pharmaceutically acceptable excipients such as diluents, binders, disintegrants, alkaline substances, antiadherants, and lubricants.

The core used according to the present invention may be pellets, beadlets or a tablet. Suitable diluents used according to the present invention are selected from microcrystalline cellulose, calcium phosphates, starch, sugar derivatives such as mannitol, lactose, sucrose and the like or a combination thereof.

Suitable binders used according to the present invention are selected from sodium alginate, povidone, methylcellulose, hydroxypropyl methylcellulose, hydroxypropyl cellulose, microcrystalline cellulose; sodium carboxymethyl cellulose, starches and the like or a combination thereof.

Suitable disintegrants used according to the present invention are selected from starch or cellulose derivatives like croscarmellose sodium, sodium starch glycolate, crospovidone, pregelatinized starch, cornstarch, sodium carboxymethyl cellulose and the like or a combination thereof.

Suitable antiadherents used according to the present invention are selected from talc, sodium lauryl sulfate, magnesium trisilicate, tribasic calcium phosphate and the like or a combination thereof Suitable lubricants used according to the present invention are selected from talc, magnesium stearate, calcium stearate, zinc stearate, stearic acid, vegetable oil, sodium lauryl sulfate, glyceryl behenate and the like or a combination thereof Suitable alkaline substances used according to the present invention are selected from sodium hydroxide, sodium bicarbonate, magnesium oxide, magnesium hydroxide, magnesium carbonate and the like, or a combination thereof.

A seal coat or protective coat, which is applied on the core, will act to physically separate the core containing acid labile drug from the acidic enteric coat, and improve stability of the formulation. In yet another embodiment of the present invention, the seal coating composition comprises polymers such as hydroxypropyl methylcellulose, hydroxypropyl cellulose, hydroxypropyl ethylcellulose, xanthan gum or a combination thereof.

The seal coating composition may further comprise plasticizer such as polyethylene glycol, dibutyl sebacate, diethyl phthalate, glycerin, glyceryl monostearate, propylene glycol, triacetin and triethyl citrate or a combination thereof.

The seal coating composition may further comprise alkaline substances selected from sodium hydroxide, sodium bicarbonate, magnesium oxide, magnesium hydroxide, magnesium carbonate and the like, or a combination thereof.

In yet another embodiment of the present invention, an enteric coating is applied onto the seal coat to delay the release of the drug from the dosage form and protect the acid labile didanosine and the composition comprises enteric polymers selected from cellulose acetate phthalate, hydroxypropyl methylcellulose phthalate, polymethacrylates, polyvinylacetate phthalate, and acrylic polymers such as Eudragit and the like or a combination thereof.

The enteric coating composition may further comprise one or more plasticizers, antiadherants and opacifiers.

Suitable opacifiers used according to the present invention are selected from titanium dioxide, or carnauba wax.

In an embodiment of the present invention, the hydroxyurea part of the composition comprises hydroxyurea and one or more pharmaceutically acceptable excipients such as diluents, binders, disintegrants, alkaline substances, antiadherants, and lubricants.

The core used according to the present invention may be pellets, beadlets or a tablet. Suitable diluents used according to the present invention are selected from microcrystalline cellulose, calcium phosphates, starches, sugar derivatives such as mannitol, lactose, sucrose and the like or a combination thereof.

Suitable binders used according to the present invention are selected from sodium alginate, povidone, methylcellulose, hydroxypropyl methylcellulose, hydroxypropyl cellulose, microcrystalline cellulose; sodium carboxymethyl cellulose, starches and the like or a combination thereof.

Suitable disintegrants used according to the present invention are selected from starch or cellulose derivatives like croscarmellose sodium, sodium starch glycolate, crospovidone, pregelatinized starch, cornstarch, sodium carboxymethyl cellulose and the like or a combination thereof.

The composition may also contain a suitable lubricant in amounts known in the art. Examples of suitable lubricants useful in the present composition include, e.g., magnesium stearate, calcium stearate, hydrogenated vegetable oil, talc, sodium stearyl fumarate, glycerol behenate or a combination thereof.

A seal coat or protective coat, which is applied on the core, will act to minimize the exposure risk of the patient to hydroxyurea powder and could improve stability of the formulation. In yet another embodiment of the present invention, the seal coating composition comprises polymers such as hydroxypropyl methylcellulose, hydroxypropyl cellulose, hydroxypropyl ethylcellulose, xanthan gum or a combination thereof.

The seal coating composition may further comprise plasticizer such as polyethylene glycol, dibutyl sebacate, diethyl phthalate, glycerin, glyceryl monostearate, propylene glycol, triacetin and triethyl citrate or a combination thereof.

The seal coating composition may further comprise alkaline substances selected from sodium hydroxide, sodium bicarbonate, magnesium oxide, magnesium hydroxide, magnesium carbonate and the like, or a combination thereof.

In yet another embodiment of the present invention, an enteric coating could be applied onto the tablet core to delay the release of the drug from the dosage form and synchronize the release of hydroxyurea with that of the other component, didanosine. The composition comprises enteric polymers selected from cellulose acetate phthalate, hydroxypropyl methylcellulose phthalate, polymethacrylates, polyvinylacetate phthalate, and acrylic polymers such as Eudragit and the like or a combination thereof.

The enteric coating composition may further comprise one or more plasticizers, antiadherants and opacifiers.

The person skilled in the art will appreciate that the pharmaceutical composition of the invention may also contain one or more other pharmacologically active agents.

Historically, Videx EC was known to exhibit a food effect (i.e., a significant reduction in bioavailability when administered with food). Within the prescription information it is stated that the product should be taken without food. In the fasted state, the exposure of didanosine at a given dose is higher and the variability of Cmax between patients and from day to day within a patient is significantly reduced.

At the start of our research, we assumed that such an interaction was very unlikely for hydroxyurea as, although the product has been marketed for more than 20 years, no such interactions have been reported in literature. Therefore, we were very surprised to find during the pharmacokinetic studies with our novel FDC therapy that hydroxyurea absorption and variability are significantly and negatively affected when the product is taken with food.

Historically, when a combination therapy of didanosine and hydroxyurea was prescribed, no instructions were issued to ensure that the products are taken concomitantly and that they should both be taken without food. As we have now found, this lack of instructions and adequate synchronizing of the administration of the two compounds must have had a negative influence on the variability of the efficacy for the combination therapy. When formulated as an FDC and accompanied with clear instructions that the product should be taken without food, this variability will probably be reduced.

Furthermore, as we have seen that the peak levels of hydroxyurea at a given dose are higher when taken in the fasted state, the potentiation of ddI can take place at a lower administered dose of hydroxyurea, thereby reducing the cost of the therapy and the risk for side effects.

Preferably the composition is formulated as ‘VS411’ (a fixed dose combination comprising didanosine and hydroxyurea). In VS411, the dosage of hydroxyurea has been lowered to reduce toxicity while maintaining its cytostatic properties and maximizing its antiviral activity. As the hydroxyurea potentiates the didanosine, a lower than usual dose of didanosine is needed in this fixed dose composition.

VS411 incorporates separate tablets of hydroxyurea (150 mg each) and enterically coated didanosine (100 mg each) into a single capsule. This allows various dosage combinations of the two agents, whilst ensuring that the two different active drugs are always taken concomitantly.

### EXAMPLES

The following examples illustrate the practice of various aspects of the present inventions. They do not limit the inventions, or the claims, which follow them.

Excipients used in the formulations of the examples are commercially available. The person skilled in the art will appreciate that methods generally known in the art may also be suitable.

### Example 1

### Manufacturing of Hydroxyurea Tablets

Tablets containing 150 mg of hydroxyurea were prepared in the following way:

3750 grams of hydroxurea, 1012.5 grams of microcrystalline cellulose, 275 grams of lactose anhydrous, 180 grams of croscarmellose sodium, 275 grams of starch 1500 were dispensed and passed through a 980 micron size screen. The screened compounds were transferred into a Winkworth drum mixer and blended for 10 minutes at 20 rpm. Subsequently, 132.5 grams of glycerol dibehenate was dispensed, passed over a 980 micron size screen and transferred into the Winkworth drum mixer. The final pre-compression mixture was prepared by blending for an additional 2 minutes at 20 rpm.

Tablets were prepared with a Manesty D3B equipped with 6.5 mm, round, normal, biconcave punches. Target tablet weight was 225 mg at a target hardness of 5 Kp.

### Example 2

### Manufacturing of ddI Enteric Coated Tablets

Enteric coated tablets containing 100 mg of ddI were prepared in the following way:

The first granulation sublot was prepared by dispensing 1000 grams of didanosine and 380 grams of lactose anhydrous and passing both through a 960 micron size screen. The screened materials were transferred into an Aeromatic Fielder GP-1 granulator and pre-blended for 2 minutes at an impeller speed of 500 rpm and granulator speed of 1500 rpm. The wet granulation procedure was started by gradually adding 152 grams of sterile water over a period of 3.5 minutes, at an impeller speed of 500 rpm and granulator speed of 1500 rpm. After water addition, granulation was continued for a further 3.5 minutes at an impeller speed of 500 rpm and granulator speed of 2000 rpm. The wet granules were transferred into an Aeromatic Fielder Strea 1 fluid bed dryer and dried at 60° C. until an LOD of lower than 1.5% was reached. Two additional sub-lots were prepared in a similar fashion.

The dried granules from all three sub-lots were subsequently passed through a 800 micron screen using a Jackson Crockatt #7.

221.8 grams of croscarmellose sodium, 443.5 grams of starch 1500, 55.4 grams of hypromellose and 27.7 grams of talc were dispensed and passed through a 960 micron size screen. The screened excipients and the screened dried granules were transferred into a Manesty drum mixer and blended for 5 minutes at 20 rpm. Subsequently, 110.9 grams of glycerol dibehenate was dispensed, passed over a 960 micron size screen and transferred into the Manesty drum mixer. The final pre-compression mixture was prepared by blending for an additional 2 minutes at 20 rpm.

Tablets were prepared with a Manesty D3B equipped with 6.5 mm, round, normal, biconcave punches. Target tablet weight was 169 mg at a target hardness of 15 Kp.

The prepared ddI tablet cores were subsequently coated with a 1.5% subcoat of Opadry White followed by a 10% enteric coating with Acryl-eze white in a Manesty Accela Coater.

### Example 3

### Manufacturing of the Fixed-Dose Composition

Two 150 mg hydroxyurea tablets, prepared as in example 1, and two 100 mg didanosine enteric coated tablets prepared as in example 2 were filled into a size 0, elongated, opaque white hard gelatin capsules. The finished product obtained the code VS411-2.

### Example 4

### Alternative Composition ddI Tablet Cores

Didanosine tablet cores were prepared along the general procedure outlined in example 2. The tablets had the following master product formula:

| PRODUCT | WEIGHT (mg) |
|---|---|
| Didanosine | 100.00 |
| Lactose Monohydrate | 38.00 |
| Pregelatinized Corn Starch | 16.00 |
| Croscaramellose Sodium | 8.00 |
| Talc | 1.00 |
| Hypromellose | 4.00 |
| Glyceryl Dibehenate | 1.00 |
| TOTAL WEIGHT OF TABLET | 168.00 |

### Example 5

### Manufacturing of Alternative Fixed-Dose Composition

Two 150 mg hydroxyurea tablets, prepared as in example 1, and two 100 mg didanosine enteric coated tablets prepared as in example 4 were filled into a size 0, elongated, opaque white hard gelatin capsules. This final product obtained the code as VS411-4

### Example 6

### Alternative Composition ddI Tablet Cores

Didanosine tablet cores were prepared along the general procedure outlined in example 2. The tablets had the following master product formula:

| PRODUCT | WEIGHT (mg) |
|---|---|
| Didanosine | 100.00 |
| Lactose Monohydrate | 38.00 |
| Pregelatinized Corn Starch | 16.00 |
| Croscaramellose Sodium | 8.00 |
| Talc | 1.00 |
| Hypromellose | 12.00 |
| Glyceryl Dibehenate | 1.00 |
| TOTAL WEIGHT OF TABLET | 176.00 |

### Example 7

### Alternative Composition ddI Tablet Cores

Didanosine tablet cores were prepared along the general procedure outlined in Example 2. The tablets had the following master product formula:

| PRODUCT | WEIGHT (mg) |
|---|---|
| Didanosine | 100.00 |
| Sodium starch glycolate | 4.20 |
| Magnesium Stearate | 1.80 |
| Silicon Dioxide | 3.75 |
| Microcrystalline Cellulose | 34.00 |
| TOTAL WEIGHT OF TABLET | 143.75 |

### Example 8

### Pharmacokinetic Studies with Hydroxyurea-Didanosine FDC Compositions

The inventors carried out a pilot Phase I, open label, randomized, single dose, 4-way crossover trial to investigate the fasted and non-fasted oral bioavailability of ddI and HU co-formulated as VS411 and administered as two different fixed dose combination (FDC) formulations, compared to commercially available ddI (Videx® EC) and HU (Hydrea®), given simultaneously.

The trial population consisted of 12 healthy adult subjects. Subjects received the study medications in four sessions (Treatment A, B, C, and D). In three of the four sessions, each subject received a single oral dose of 400 mg ddI and 600 mg HU while, due to the commercial availability of Hydrea as only 500 mg capsules, each subject received 500 mg instead of 600 mg HU during one session.

Treatment A consisted of two capsules of VS411 as formulation VS411-2, a FDC formulation of 200/300 mg ddI/HU (composition as in Example 3), administered under fasted conditions. Treatment B consisted of two capsules of VS411 as formulation VS411-4, a FDC formulation of 200/300 mg ddI/HU, administered under fasted conditions (composition as in example 5). Treatment C consisted of one capsule of 400 mg Videx® EC and one capsule of 500 mg Hydrea® administered under fasted conditions.

Randomization of Treatments A, B, and C in Sessions I, II, and III was done in such a way that in each session four out of 12 subjects received the same treatment and each subject received a different treatment in each session. After Session III an interim pharmacokinetic analysis was performed to select formulation VS411-2 or VS411-4 for further investigation in Session IV.

In Session IV each subject received Treatment D, which consisted of two capsules of VS411 as formulationVS411-2 administered after a high-fat breakfast. There was a washout period of at least one week between subsequent intakes of trial medication. In each session, a full pharmacokinetic profile of ddI and HU was determined up to 24 hours postdose. The short-term safety and tolerability was assessed on an ongoing basis. The results were as follows:

TABLE 1
Summary of results: Pharmacokinetic analysis for ddI in FDC compositions.

| Pharmacokinetics of ddI | | | | |
|---|---|---|---|---|
| (mean ± SD, tₘₐₓ: median | | | Videx EC + | |
| [range]) | VS411-2 | VS411-4 | Hydrea | VS411-2, fed |
| n | 12 | 12^{a} | 12 | 12^{b} |
| Cₘₐₓ, ng/mL | 989.4 ± 597.3 | 1147 ± 912.3 | 1293 ± 526.6 | 497.1 ± 460.8 |
| t_{lag}, h | 0.5 (0.0-3.0) | 0.75 (0.0-16.0) | 0.5 (0.0-1.5) | 3.0 (0.5-16.0) |
| tₘₐₓ, h | 3.0 (1.0-4.0) | 4.0 (2.5-24.0) | 2.0 (1.0-5.0) | 7.5 (2.5-24.0) |
| AUCₗₐₛₜ, ng · h/mL | 3273 ± 1363 | 3806 ± 2247 | 3672 ± 1023 | 2142 ± 1582 |
| AUCₓ, ng · h/mL | 3325 ± 1365 | 3868 ± 2244 | 3717 ± 1025 | 2970 ± 1499 |
| t_{1/2term}, h | 1.775 ± 0.3175 | 1.941 ± 0.9520 | 1.729 ± 0.3402 | 1.520 ± 0.1004 |

LSmean ratio (90% CI), %

| | VS411-2 vs | VS411-4 vs | | VS411-2, fed vs |
|---|---|---|---|---|
| | Videx EC + Hydrea | Videx EC + Hydrea | — | VS411-2, fasted |
| n | 12 vs 12 | 12 vs 12^{c} | — | 12 vs 12^{d} |
| Cₘₐₓ | 71.30 (53.41-95.17) | 73.24 (49.37-108.7) | — | 40.78 (23.84-69.75)* |
| AUCₗₐₛₜ | 84.28 (68.09-104.3) | 92.82 (63.65-135.3) | — | 52.49 (29.63-93.01) |
| AUCₓ | 84.75 (68.77-104.5) | 93.70 (64.77-135.6) | — | 84.29 (54.27-130.9) |

^{a}n = 11 for AUCₗₐₛₜ, AUCₓ and t_{1/2term}
^{b}n = 11 for AUCₗₐₛₜ and n = 7 for AUCₓ and t_{1/2term}
^{c}n = 11 vs 11 for AUCₗₐₛₜ and AUCₓ
^{d}n = 11 vs 11 for AUCₗₐₛₜ n = 7 vs 7 for AUCₓ
*Statistical significant difference

The results of this exploratory study showed that the Cmax of ddI was decreased by 29% and 27% for formulations VS411-2 and VS411-4, respectively, compared to the commercial formulation Videx®. AUCₗₐₛₜ was decreased by 16% and 7%. However, the levels of didanosine reached were above the minimum effective concentration for a sufficiently long period of time. As inter-subject variability was larger for the VS411-4 formulation, the VS411-2 formulation was selected for a food effect comparison. Food decreased ddI exposure significantly and increased inter-subject variability. Thus VS411-2 will be more effective if taken without food.

TABLE 2
Summary of results: Pharmacokinetic analysis of hydroxyurea in FDC
compositions.

| | Descriptive statistics, linear mixed effects modeling, | | | |
|---|---|---|---|---|
| Statistical methods | for tmax: | | | |
| Pharmacokinetics of HU | univariate test (two-sided Wilcoxon signed-ranks test) | | | |
| (mean ± SD, tₘₐₓ: median | | | Videx EC + | |
| [range]) | VS411-2 | VS411-4 | Hydrea | VS411-2, fed |
| n | 12 | 12 | 12 | 12 |
| Cₘₐₓ, ng/mL | 16010 ± 2742 | 15460 ± 3453 | 13530 ± 2305 | 10770 ± 2150 |
| tₘₐₓ, h | 0.75 (0.5-2.0) | 1.0 (0.5-3.0) | 1.0 (0.5-2.0) | 1.75 (0.5-5.0) |
| AUCₗₐₛₜ, ng · h/mL | 68440 ± 6300 | 71220 ± 8200 | 56810 ± 7444 | 63620 ± 10050 |
| AUCₓ, ng · h/mL | 69010 ± 6291 | 71840 ± 8295 | 57200 ± 7467 | 64220 ± 10160 |
| t_{1/2term} h | 3.352 ± 0.2935 | 3.330 ± 0.3652 | 3.236 ± 0.3410 | 3.291 ± 0.3331 |

LSmean ratio (90% CI), %^{a}

| | VS411-2 vs | VS411-4 vs | | VS411-2, fed vs |
|---|---|---|---|---|
| | Videx EC + Hydrea | Videx EC + Hydrea | — | VS411-2, fasted |
| n | 12 vs 12 | 12 vs 12 | — | 12 vs 12 |
| Cₘₐₓ | 98.42 (90.19-107.4) | 94.04 (82.48-107.2) | — | 66.97 (57.91-77.44)* |
| AUCₗₐₛₜ | 100.8 (97.22-104.5) | 104.6 (99.97-109.5) | — | 92.22 (88.05-96.59)* |
| AUCₓ | 100.9 (97.48-104.5) | 104.8 (100.2-109.7) | — | 92.31 (88.16-96.66)* |

^{a}For statistical analysis parameters were dose normalized to 500 mg
*Statistical significant difference

Cmax and AUCs of HU in the VS411-2 and VS411-4 formulations were almost identical to those of the commercial formulation Hydrea®. However, unexpectedly, food decreased HU exposure significantly and increased inter-subject variability. Thus VS411-2 will be more effective if taken without food.

In summary, this pharmacokinetic study shows that the novel fixed dose combination product provides sufficiently high plasma levels of both didanosine and hydroxyurea to be effective. Additionally, it shows how important it is to administer the combination in the fasted state as a FDC product, thereby ensuring adequate drug exposures and reducing inter-subject variability and providing a means to lower the dose of both active ingredients.

1. A fixed-dose pharmaceutical composition comprising hydroxyurea and didanosine.

2. A composition according to claim 1, comprising 200 to 1000 mg hydroxyurea and 200 to 400 mg didanosine.

3. A composition according to claim 1, comprising 300 mg hydroxyurea and 200 mg didanosine.

4. A composition according to claim 1, comprising 637.5 mg hydroxyurea and 325 mg didanosine.

5. A composition according to claim 1, comprising 825 mg hydroxyurea and 250 mg didanosine.

6. A composition according to one or more of the claims 1-5 in the form of a capsule comprising one or more separately formulated pharmaceutical dosages of hydroxyurea and/or didanosine.

7. A composition according to one or more of the claims 1-6, in which the one or more separately formulated pharmaceutical dosages consist of one or more tablets comprising hydroxyurea and/or didanosine.

8. A composition according to one or more of claims 1-7 in which the hydroxyurea is present in a tablet in compressed form.

9. A composition according to one or more of claims 1-7, in which the tablet containing didanosine is enteric coated.

10. A composition according to one or more of claims 1-9 which exhibits decreased plasma levels for both active ingredients when taken with food.

11. A composition according to one or more of claims 1-9 which exhibits maximum plasma levels for both active ingredients when taken without food.

12. A method of manufacturing a pharmaceutical composition according to one or more of the claims 1-11.

13. A composition according to one or more of the claim 1-12 which is administered to a patient in need thereof in a single dose once daily.

14. A pharmaceutical combination comprising hydroxyurea and didanosine according to one or more of the claim 1-13, which is taken without food.

15. A pharmaceutical combination comprising hydroxyurea and didanosine according to one or more of the claims 1-14, which when administered without food synchronizes the peak plasma levels for both compounds to within a 2.5 hour period.

16. A pharmaceutical formulation comprising hydroxyurea and didanosine according to one or more of the claims 1-14, which when administered without food provides peak plasma levels for both compounds not more than 2.5 hours apart from one another.

17. A pharmaceutical formulation comprising hydroxyurea and didanosine according to one or more of the claims 1-16, characterized in that when tested in an USP dissolution apparatus nr II, with 900 ml dissolution medium phosphate buffer at pH=6.8, and an impeller speed of 50 rpm, that the amount of each active ingredient released within 45 minutes is equal to or more than 75% (Q) of the labelled content for each active ingredient.

18. A method of treatment of a patient suffering from an infection caused by a human immunodeficiency virus comprising administering to a diseased patient a pharmaceutical comprising a combination of didanosine and hydroxyurea, characterized in that a pharmaceutical composition according to one or more of the claims 1-17 is administered as a single dose.
